(19) United States
(12) Patent Application Publication
(10) Pub. No.: US 2008/0147046 A1
(43) Pub. Date: Jun. 19, 2008

(54) CATHETER HAVING A SPIRALLY SLICED TUBE

(76) Inventor: Benjamin David McDaniel, Irvine, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/612,838

(22) Filed: Dec. 19, 2006

(51) Int. Cl. A61M 25/14

(52) U.S. Cl. 604/530

(57) ABSTRACT

A elongate tubular catheter uses spirally sliced tubes to provide flexible support having pushability in a number of roles. The spirally sliced tube may be used in place of a stiffening tube thereby providing a catheter that can have variable degrees of flexibility along its length. Alternatively, the spirally sliced tube may be used in place of a compression wire or other puller wire sleeve. The spirally sliced tubes may also be used as the inner or outer wall of an irrigation lumen within the catheter. The flexibility of the catheter can be easily modified during manufacture by varying the pitch angle of the spiral slice. Flexibility along the length of the catheter can also be modified by using one or more spirally sliced tubes or tubes in which only portions are spirally sliced.

### FIELD OF THE INVENTION

The present invention relates to a medical device for use in the vessel of a patient wherein a portion of the catheter is comprised of a spirally sliced tube. More particularly, the catheter uses a spirally sliced polymer or shape metal memory tube to provide a flexible yet pushable means of providing flexible support and/or enclosing one or more puller wires and/or irrigation lumens.

### BACKGROUND OF THE INVENTION

Many abnormal medical conditions in humans and other mammals have been associated with disease and other aberrations along the lining or walls that define several different body spaces. In order to treat such abnormal conditions of the body spaces, medical device technologies adapted for delivering various therapies to the body spaces using the least invasive means possible.

As used herein, the term “body space,” including derivatives thereof, is intended to mean any cavity within the body which is defined at least in part by a tissue wall. For example, the cardiac chambers, the uterus, the regions of the gastrointestinal tract, and the arterial or venous vessels are all considered illustrative examples of body spaces within the intended meaning.

The term “vessel,” including derivatives thereof, is herein intended to mean any body space which is circumscribed along a length by a tubular tissue wall and which terminates at each of two ends in at least one opening that communicates externally of the body space. For example, the large and small intestines, the vas deferens, the trachea, and the fallopian tubes are all illustrative examples of vessels within the intended meaning. Blood vessels are also herein considered vessels, including regions of the vascular tree between their branch points. More particularly, the pulmonary veins are vessels within the intended meaning, including the region of the pulmonary veins between the branched portions of their ostia along a left ventricle wall, although the wall tissue defining the ostia typically presents uniquely tapered lumenal shapes.

One means of treating body spaces in a minimally-invasive manner is through the use of catheters to reach internal organs and vessels within a body space. Electrode catheters have been in common use in medical practice for many years. They are used to stimulate and map electrical activity in the heart and to ablate sites of aberrant electrical activity. In use, the electrode catheter is inserted into a major vein or artery, e.g., the femoral artery, and then guided into the chamber of the heart which is of concern. Within the heart, the ability to control the exact position and orientation of the catheter tip is critical and largely determines how useful the catheter is.

Steerable catheters are generally well known. For example, U.S. Pat. No. RE 34,502 describes a catheter having a control handle comprising a housing having a piston chamber at its distal end. A piston is mounted in the piston chamber and is afforded lengthwise movement. The proximal end of the catheter body is attached to the piston. A puller wire is attached to the housing and extends through the piston and through the catheter body. The distal end of the puller wire is anchored in the tip section of the catheter to the sidewall of the catheter shaft. In this arrangement, lengthwise movement of the piston relative to the housing results in deflection of the catheter tip section.

In bidirectional steerable catheters, a pair of puller wires extends through a lumen in the main portion of the catheter shaft and then into opposing off-axis lumens in a deflectable tip section where the distal end of each puller wire is attached to the outer wall of the deflectable tip. Pulling one wire in a proximal direction causes the tip to deflect in the direction of the off axis lumen in which that wire is disposed.

In other designs, the puller wires are attached to opposite sides of a rectangular plate that is fixedly mounted at its proximal end and extends distally within a lumen in the tip section. In this arrangement, pulling one of the wires proximally causes the rectangular plate to bend in the direction of the side to which the pulled puller wire is attached, thereby causing the entire tip section to deflect. In most designs stainless steel compression coils are commonly used in conjunction with the puller wires to reduce flexure of a region of the catheter when the puller wires are tensioned.

The steerability of the catheter is directly related to the flexibility of the catheter. It would therefore be desirable to have a catheter that is sufficiently flexible to be easily steerable but that is also pushable within the body space.

In certain applications, it is desirable to have the ability to inject and/or withdraw fluid through the catheter. This is accomplished by means of an irrigated tip catheter. One such application is a cardiac ablation procedure for creating lesions that interrupt errant electrical pathways in the heart.

A typical ablation procedure involves the insertion of a catheter having a tip electrode at its distal end into a heart chamber. A reference electrode is provided, generally taped to the skin of the patient. RF (radio frequency) current is applied to the tip electrode, and current flows through the media that surrounds it, i.e., blood and tissue, toward the reference electrode. The distribution of current depends on the amount of electrode surface in contact with the tissue as compared to blood, which has a higher conductivity than the tissue. Heating of the tissue occurs due to its electrical resistance. The tissue is heated sufficiently to cause cellular destruction in the cardiac tissue resulting in formation of a lesion within the cardiac tissue that is electrically non-conductive.

The tissue is heated sufficiently to cause cellular destruction in the cardiac tissue resulting in formation of a lesion within the cardiac tissue that is electrically non-conductive. During this process, heating of the electrode also occurs as a result of conduction from the heated tissue to the electrode itself. If the electrode temperature becomes sufficiently high, possibly above 60 degrees centigrade, a thin transparent coating of dehydrated blood protein can form on the surface of the electrode. If the temperature continues to rise, this dehydrated layer can become progressively thicker resulting in blood coagulation on the electrode surface. Because dehydrated biological material has a higher electrical resistance than endocardial tissue, impedance to the flow of electrical energy into the tissue also increases. If the impedance increases sufficiently, an impedance rise occurs and the catheter must be removed from the body and the tip electrode cleaned.

One method used to reduce the negative affects of heating is to irrigate the ablation electrode, e.g., with physiologic saline at room temperature, to actively cool the ablation electrode instead of relying on the more passive physiological cooling of the blood. Because the strength of the RF current is no longer limited by the interface temperature, current can be increased. This results in lesions which tend to be larger and more spherical, usually measuring about 10 to 12 mm. In addition to irrigation flow during ablation, a maintenance flow, typically at a lower flow rate, is required throughout the procedure to prevent backflow of blood flow into the coolant passages. Thus, it is necessary to provide for catheters that are flexible, steerable and yet contain the necessary structure to provide lumens for irrigation.

Another issue for catheters arises when they are used in RMT systems. In remote magnetic technology (RMT) systems, magnets external to the patient are used to product magnetic fields in the patient that can guide a catheter such as a catheter for ablation. Catheters used for this purpose must have a high degree of flexibility so that the magnetic fields can properly guide the device through the tortuous anatomy of the patient. It would, therefore be desirable to have a catheter that is highly flexible but yet has the proper magnetic characteristics for use in an RMT system.

Additionally, catheters that are used during magnetic resonance imaging (MRI) procedures need to be non-magnetic. Due to the powerful electromagnets used to align the nuclei of hydrogen atoms in the water content of the human body the use of catheters having magnetic properties is prohibited. Therefore, it would be desirable to have a flexible, steerable non-magnetic catheter for use during MRI procedures.

### SUMMARY OF THE INVENTION

The present invention generally relates to a catheter having a spirally sliced tube in place of one or more solid tubular stiffening tubes or metal compression coils in a steerable catheter. More specifically, the present invention is a catheter comprising an elongate tubular member having a proximal end and a distal end and a lumen defined by the inner diameter of the tubular member and having an elongate spirally sliced tubular member disposed within the lumen of the elongated tubular member. The catheter can have the spirally sliced tubular member disposed immediately within the inner diameter of the elongate tubular member in order to provide a controllable stiffening tube. Alternatively, the catheter can use the elongate tubular member to receive one or more puller wires for steering the distal end of the catheter. Additionally, the catheter may have an irrigation lumen disposed with the lumen of the elongate tubular member wherein the irrigation lumen comprises an outer wall and an inner wall. Either the inner wall or the outer wall may be a spirally sliced catheter.

A catheter in accordance with the present invention exhibits reduced stiffness in comparison to metallic compression coils or solid tubular constructions.

Furthermore, a catheter in accordance with the present invention, particularly one using the polymeric tube, has little or no interaction with magnetic or electrical field in comparison to those having compression coils made of other metals.

Additionally, catheters in accordance with the present invention exhibit increased resilience and robustness during handling and assembly.

Still further, the stiffness of a catheter made in accordance with the present invention can have varying degrees of stiffness by varying the pitch and/or stopping the spiral slice at a specific points along the longitude of the polymer tube.

### BRIEF DESCRIPTION OF THE DRAWINGS

FIG. 1A is a side elevational view of one embodiment of a spirally sliced tube for use in a catheter in accordance with the present invention.

FIG. 1B is a side elevational view of a further embodiment of a spirally sliced tube for use in a catheter in accordance with the present invention.

FIG. 1C is a side elevational view of an additional embodiment of a spirally sliced tube for use in a catheter in accordance with the present invention.

FIG. 2 is a cross sectional view of the spirally sliced tube for use in a catheter in accordance with the present invention.

FIG. 3 depicts a perspective view of an RF ablation catheter in accordance with the present invention.

FIG. 4 is a perspective partial cross-sectional view of the flexible tubular section of the catheter of FIG. 3.

FIG. 5 is a longitudinal cross-sectional view of the flexible tubular section of the catheter of FIG. 3.

FIG. 6 is a cross-sectional view of the tubular section of the catheter of FIG. 5.

FIG. 7 is a longitudinal cross-section view of the flexible tubular section of an irrigation catheter in accordance with the present invention.

FIG. 8 is a cross-sectional view of the irrigation catheter of FIG. 7.

### DETAILED DESCRIPTION OF THE INVENTION

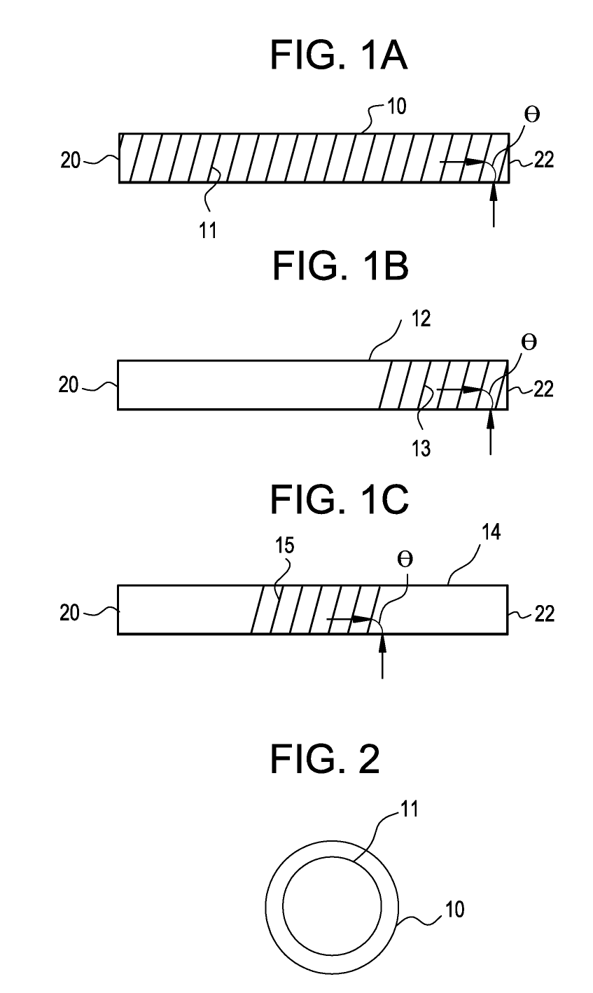

FIG. 1A depicts a spirally sliced tube for use in a catheter in accordance with the present invention. Elongate tube 10 has a spiral slice 11 cut through the wall of the tube 10 extending from proximal end 20 to distal end 22 thereby enabling tube 10 to more easily flex in the direction perpendicular to the axis of tube 10. FIG. 1B depicts a variation in which tube 12 has a spiral slice 13 cut through the wall of tube 12 extending distally from proximal end 20 to distal end 22. Tube 12 may be reversed so that the spirally sliced area extends from the proximal end to a point proximal the distal end. FIG. 1C depicts a further variation in which tube 14 has a spiral slice 15 cut through the wall of tube 14 from a point distal the proximal end 20 to a point proximal the distal end 22. Various combinations of tubes 10, 12, and 14 can be used to provide various sections of a catheter in accordance with the present invention with differing degrees of flexure along the length of the catheter as will be described in more detail herein.

Tube 10 can be made from a variety of polymers including PEEK™ polymer from Victrex plc, polyamide, polyurethane, nylon, and PEBAX among others. VICTREX™ PEEK™ polymer, is a repeat unit that comprises of oxy-1, 4-phenyleneoxy-1, 4-phenylene-carbonyl-1, 4-phenylene. PEEK is a linear aromatic polymer that is semi-crystalline.

Tubes 10, 12 and 14 may also be made of a shape memory metal or alloy. A shape memory alloy, also known as shape memory metal, is a material that returns to an original geometry after deformation from its “original” formation. A shape memory alloy may return to its original geometry by itself during heating (one-way effect) or, at higher ambient temperatures, simply during unloading due to pseudo-elastic or superelastic properties. These properties are due to a temperature-dependent martensitic phase transformation from a low-symmetry to a highly symmetric crystallographic structure. Three types of shape memory alloys are copper-zinc-aluminum, copper-aluminum-nickel and nickel-titanium (NiTi) alloys although any type of shape-memory metal or alloy can be used. Some examples of shape-memory metals are set forth below:
Ag—Cd 44/49 at. % Cd
Au—Cd 46.5/50 at. % Cd
Cu—Al—Ni 14/14.5 wt. % Al and 3/4.5 wt. % Ni
Cu—Sn approx. 15 at. % Sn
Cu—Zn 38.5/41.5 wt. % Zn
Cu—Zn—X (X=Si, Sn, Al) a few wt. % of XXX
In—Ti 18/23 at. % Ti
Ni—Al 36/38 at. % Al
Ni—Ti 49/51 at. % Ni
Fe—Pt approx. 25 at. % Pt
Mn—Cu 5/35 at. % Cu
Fe—Mn—Si
Pt alloys
Co—Ni—Al
Co—Ni—Ga

FIG. 2 depicts the cross-section of a spirally sliced tube 10 (and also tubes 12 and 14 at the sliced portion). Slice 11 is completely cut through the wall of tube 10.

The inner and outer dimensions of tubes 10, 12 and 14 can vary depending on the application within a catheter, the degree of flexure necessary and the amount of pushability desired. Preferably, the inner dimension of tubes 10, 12 and 14 are between 0.1 and 10 mm with an outer dimension of between 0.101 and 15 mm. The thickness should preferably be between 0.001 and 14.9 mm. A preferred embodiment has an inner diameter of approximately 0.45″ and an outer diameter of approximately 0.45″.

The pitch angle, θ, of the spiral slice may also be varied depending on the amount of flexure desired and the degree of pushability desired. Increasing the pitch angle, θ, will make the tube more flexible while decreasing the pitch angle will create a tube with more support and less flex. Preferably, the pitch angle is between 0 and 30 degrees. The pitch angle may be varied on the same tube.

Tubes 10, 12, and 14 can be used in any combination of quantity, position and pitch of various sliced segments within a catheter in order to impart different characteristics into the catheter. For example, a first spirally sliced tube 10 with a pitch angle θ₁ and a second spirally sliced tube 10 with a different pitch angle θ₂, wherein θ₁>θ₂, could be placed in a catheter so that the first tube is distal to the second. This arrangement would provide a catheter with more flexure at the distally than proximally. Myriad combinations of tubes with different angles and spirally sliced portions can be created in order to customize catheter flexure.

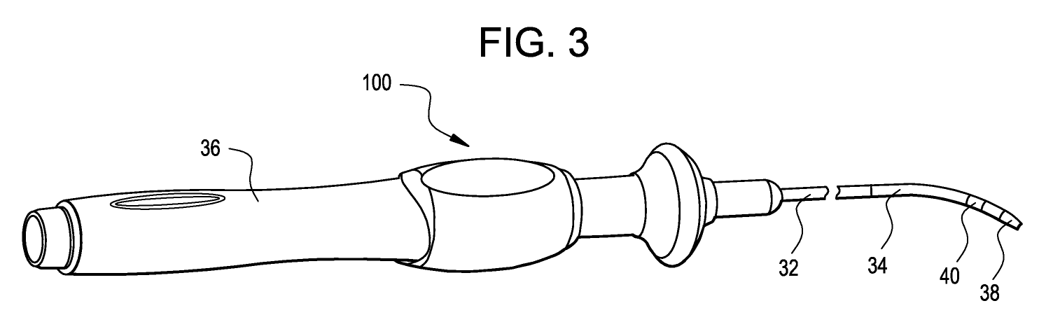

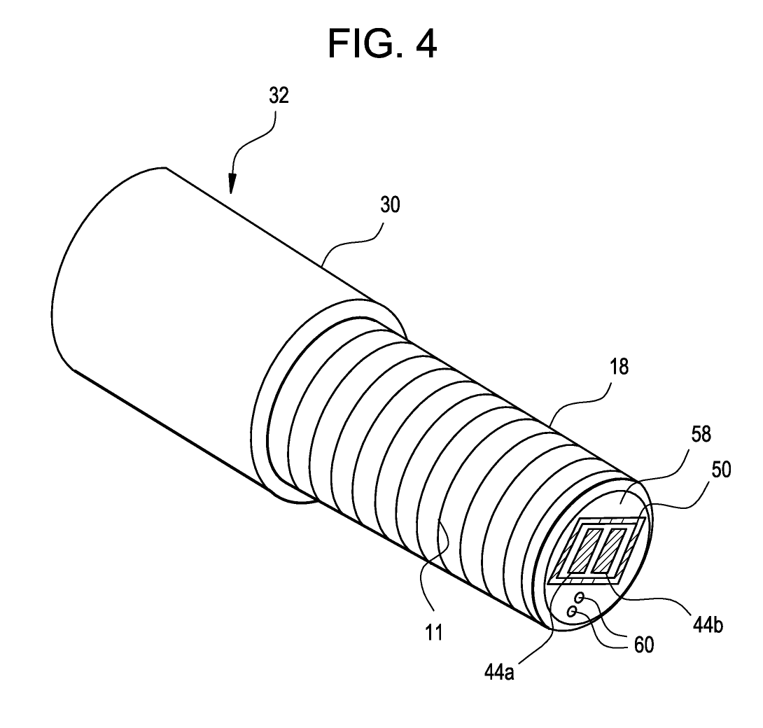

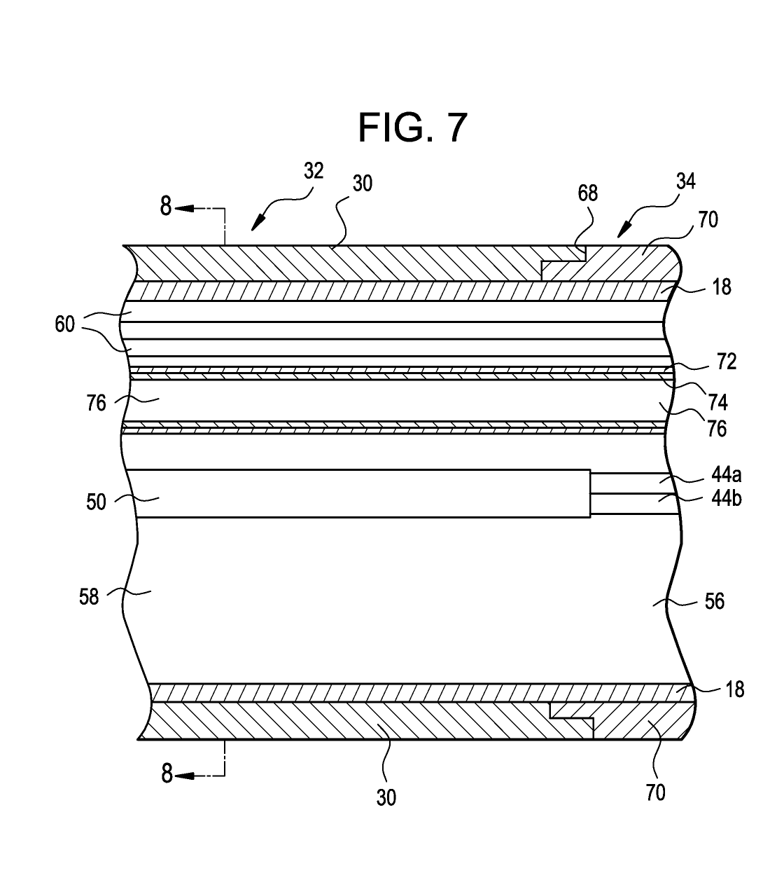

FIG. 3 is a perspective view of an embodiment of a catheter in accordance with the present invention. As shown in FIG. 3, a preferred catheter 100 comprises an elongated tubular catheter body having a proximal section 32, a distal tip section 34 and a control handle 36 at the proximal end of the proximal section 32. Tip electrode 38 and ring electrode 40 may be added at or near distal tip section 34 so as to provide a source of ablation energy if the desired device is an RF ablation catheter. Otherwise the distal tip section 34 may be varied in order to provide different functionality for the catheter. For example, distal tip section could contain irrigation ports connected to an irrigation lumen (as depicted in FIGS. 7 and 9) in order to provide a flow of irrigating fluid from a source external to the body space of the patient.

As shown in FIGS. 4-6, the proximal section 32 comprises an elongated tubular construction having a single axial or central lumen 58. The proximal section 32 is flexible but substantially non-compressible along its length. Proximal section 32 can be made of any suitable construction and made of any suitable material. The preferred construction comprises an outer wall 30 made of polyethylene or PEBAX and an inner wall 18. The outer wall 30 may also comprise an imbedded braided mesh of stainless steel or similar material to increase torsional stiffness so that when control handle 36 is rotated the tip section 34 will rotate in a corresponding manner.

The overall length of the length of the catheter will vary according to its application for use but a preferred length is between approximately 90 and 120 cm and more preferably between approximately 100 and 110 cm. The outer diameter of the proximal section 32 is also a design characteristic that varies according to the application of the catheter but is preferably less than approximately 8 French (Fr). Inner wall 18 comprises a spirally-sliced tube (also referred to as a spirally-sliced tubular member) 10 and is sized so that the outer diameter is about the same size or slightly smaller than the inner diameter of outer wall 30 thereby providing additional stiffness which can be controlled by the pitch angle of the cut as described above.

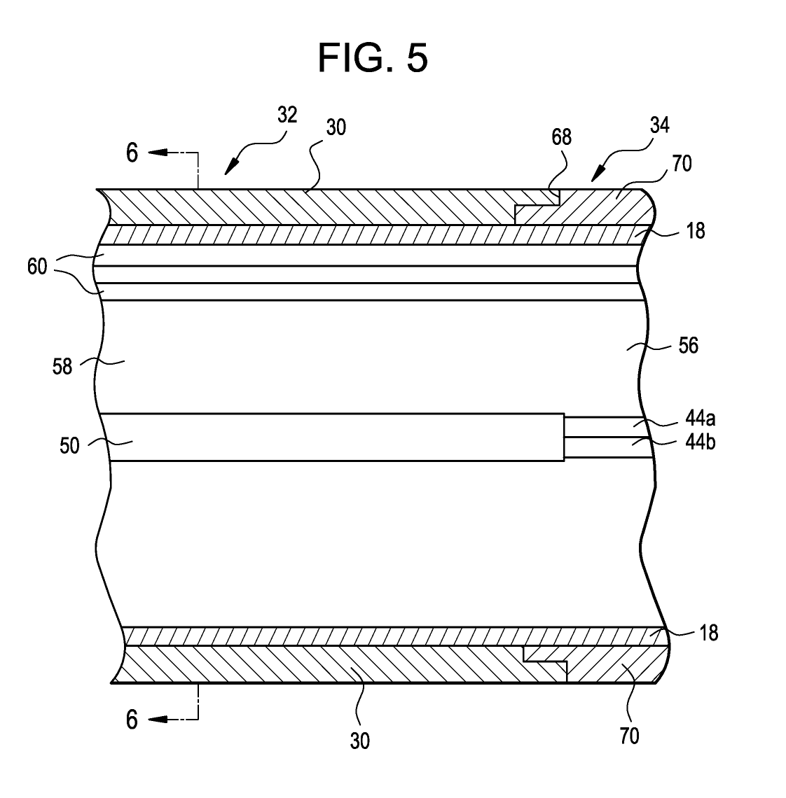

FIG. 5 is a cross-sectional depiction of the catheter along the longitudinal axis at the transition between the proximal section 32 and the distal section 34. Outer wall 30 transitions to flexible tubing 70 having lumen 56 extending therethrough, although additional lumens can be included if desired as discussed below with respect to an irrigation lumen. Flexible tubing 74 is made of a suitable non-toxic material that is generally more flexible than the outer wall 30 of the proximal section 32. A presently preferred material for flexible tubing 74 is polyurethane although other materials such as nylon may also be used. The outer diameter of the distal section 34 is preferably no greater than about 8 Fr and is more preferable 6½ Fr or less. In the embodiment shown, the distal section 34 and the proximal section 32 are separate structures that have been fixedly attached to each other. It is understood that the distal section 34 and the proximal section 32 could be formed as a unitary structure as desired. Additionally, in FIGS. 4-8 inner wall 18 may be formed of any combination of spirally sliced tubes 10, 12 and 14 placed along the length of the catheter.

In an RF ablation catheter, tip electrode 38 and ring electrode 40 are each electrically connected to a separate lead wire 60. Each lead wire 60 extends from the control handle 36 through the lumen 58 in the proximal section 32 and through lumen 56 in distal section 34 to tip electrode 38 and ring electrode 40. The proximal end of each lead wire 60 is connected to an appropriate connector (not shown) in the control handle 36 which can be plugged into a suitable source of rf energy.

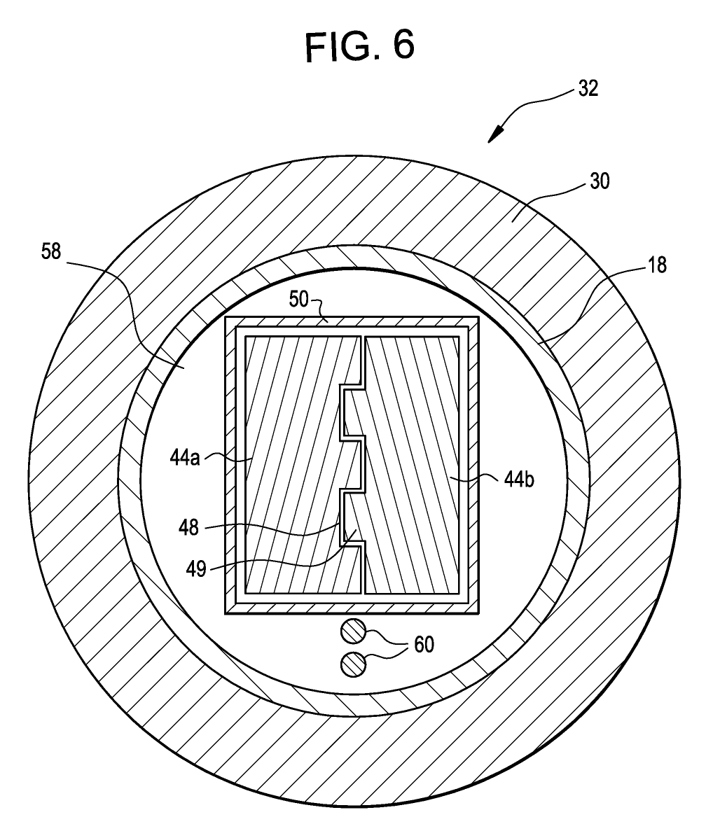

In a bi-directional rf ablation catheter a pair of puller wires 44a and 44b extend through the through the lumen 58 in the proximal section 32 and through lumen 56 in distal section 34. The puller wires are made of any suitable material such as stainless steel or Nitinol. Preferably, each puller wire 44 is covered with a lubricious coating such as PTFE or a similar material. Each puller wire 44 extends from the control handle 36 to near the tip of distal section 34. Puller wires 44 may be slidably mated to each other along a portion of their length in various manners such as that depicted in FIG. 6 in which puller wires 44a and 44b are interlocked. At their distal end the two puller wires 44a and 44b are fixedly attached to each other at a joint (not shown) by soldering, welding, bonding or similar method. Puller wires 44 can have any desired cross-sectional shape, e.g., round, rectangular, square, ellipsoidal, etc. and the cross-sectional shape of one wire does not need to be the same as the other. There are several ways in which the puller wires can be mated along their length including the generally rectangular notches 48 of puller wire 44a that mate with rectangular ribs 49 of puller wire 44b.

A sleeve 50 is provided that surrounds the puller wires to keep them in a closely adjacent relationship. Sleeve 50 may be made of any suitable material, e.g., polyamide or polyurethane or comprise a compression coil. Alternatively, sleeve 50 may also comprise one or more a spirally sliced tubes 10, 12 and 14 that provides the catheter designer with an ability to change the characteristics of the response of the catheter to the puller wires 44a and 44b by replacing the compression coil with a spirally-sliced tube or tubes 10, 12, or 14.

Examples of other suitable control handles that can be used with the present invention are described in U.S. Pat. No. 6,123,699, 6,171,277, 6,183,463 and 6,198,974 the disclosure of which are hereby incorporated by reference. Additional configurations of puller wires 44 and gearing within the control handle may be used such as those disclosed in U.S. Pat. No. 7,077,823 which is also hereby incorporated by reference.

An alternative embodiment of a catheter in accordance with the present invention would provide a standard (non-spirally sliced) stiffening tube in place of the spirally-sliced tube 10 for inner wall 18. The only spirally-sliced tube would be used for the sleeve 50. The stiffening tube for inner wall 18 could be made of could be made of any suitable material, but would preferably be made of polyimide or nylon.

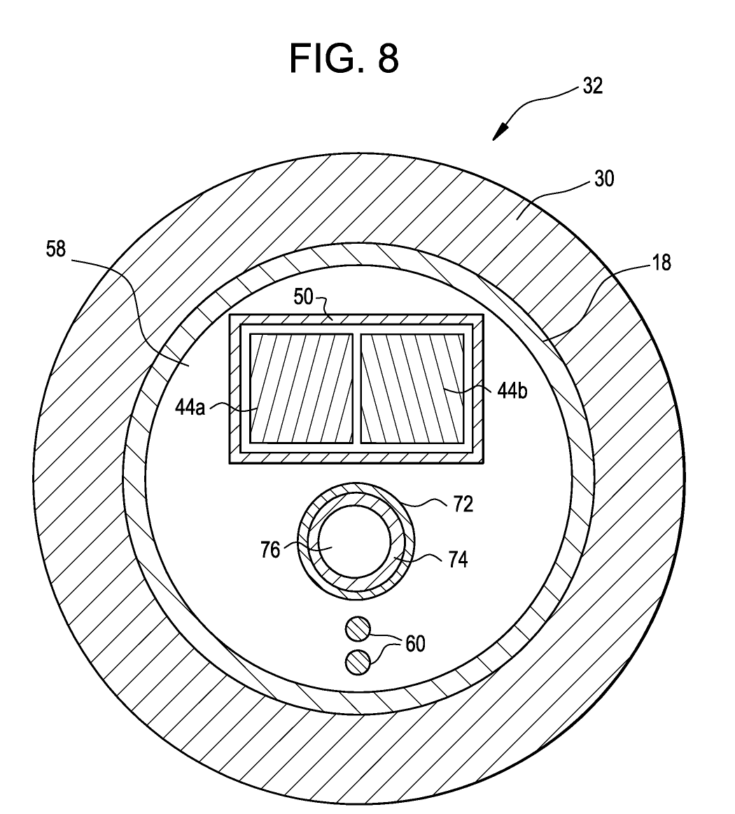

The spirally-sliced tube 10, 12 or 14 may also be used to inside and/or outside an irrigation lumen. Placement outside an irrigation lumen would increase the hoop strength of a low durometer polymer tubing used as an irrigation lumen while also being flexible. FIGS. 7 and 8 depict the longitudinal cross-section and transverse cross-section of a catheter 100 having an irrigation lumen 76 within main lumen 58. The irrigation lumen 76 has a two-piece construction having an outer irrigation lumen wall 72 and an inner irrigation lumen wall 74. In one embodiment the inner irrigation lumen wall 74 is made from one or more spirally-sliced tubes 10, 12 or 14 thereby providing an irrigation lumen that prevents kinking, increases compressive strength without reducing flexibility and prevents collapse during application of a vacuum. In this embodiment outer irrigation lumen wall 72 is made of any suitable material but is preferably made of a low durometer polymer. In another embodiment the order is reversed an the outer irrigation lumen wall 72 is made from one or more spirally-sliced tubes 10, 12 or 14 while inner irrigation lumen wall 74 is made of any suitable material but is preferable a low durometer polymer. This provides an irrigation lumen with increased hoop/burst strength without increasing the stiffness and thereby decreasing the flexibility of the catheter. Irrigation lumen 76 runs from a connection in control handle 36 through the proximal section 32 and the distal section 34 to at or near the tip where it is in fluid communication with one or more irrigation ports (not shown).

The preceding description has been presented with reference to presently preferred embodiments of the invention. Workers skilled in the art and technology to which this invention pertains will appreciate that alterations and changes in the described structure may be practiced without meaningfully departing from the principal, spirit and scope of this invention.

Accordingly, the foregoing description should not be read as pertaining only to the precise structures described and illustrated in the accompanying drawings, but rather should be read consistent with and as support to the following claims which are to have their fullest and fair scope.

What is claimed is:

1. A catheter for use in a vessel comprising:
an elongate tubular member having a proximal end and a distal end and a lumen defined by the inner diameter of the tubular member; and,
an elongate spirally-sliced tubular member disposed within the lumen of the elongated tubular member.

2. The catheter of claim 1 wherein the spirally sliced tubular member has an outer diameter substantially the same size as the inner diameter of the elongate tubular member.

3. The catheter of claim 1 wherein the spirally sliced tubular member has an outer diameter smaller than the inner diameter of the elongate tubular member adapted to receive one or more puller wires for steering the distal end of the catheter.

4. The catheter of claim 1 further comprising an irrigation lumen disposed with the lumen of the elongate tubular member wherein the irrigation lumen comprises an outer wall and an inner wall.

5. The catheter of claim 4 wherein the inner wall of the irrigation lumen is an elongate spirally sliced tubular member.

6. The catheter of claim 4 wherein the outer wall of the irrigation lumen is an elongate spirally sliced tubular member.

7. The catheter of claim 1 wherein the elongate spirally sliced tubular member is a polymer.

8. The catheter of claim 7 wherein the polymer is selected from the group consisting of polyamide, polyurethane, nylon, PEBAX and PEEK polymers and blends thereof.

9. The catheter of claim 1 wherein the elongate spirally sliced tubular member is a shape memory metal.

10. The catheter of claim 9 wherein the shape memory metal is Nitinol.

11. The catheter of claim 1 wherein the elongate spirally sliced tubular member has a pitch angle, θ, for the spiral slice that varies along the length of the tubular member.

12. The catheter of claim 1 wherein the elongate spirally sliced tubular member has a pitch angle, θ, for the spiral slice of less than approximately 30 degrees.

13. The catheter of claim 1 wherein the elongate spirally sliced tubular member comprises a slice cut portion and a solid portion.

14. The catheter of claim 1 wherein the elongate spirally sliced tubular member comprises a plurality of spirally sliced tubular members.

15. A catheter for use in a vessel comprising:
an elongate tubular member having a proximal end and a distal end and a lumen defined by the inner diameter of the tubular member; and,
an irrigation lumen disposed within the lumen of the elongated tubular member wherein said irrigation lumen comprises an inner wall and an outer wall and wherein one of the inner wall or the outer wall is a spirally sliced tubular member.

16. The catheter of claim 15 wherein the inner wall is a spirally sliced tubular member.

17. The catheter of claim 15 wherein the outer wall is a spirally sliced tubular member.
